(19) United States
(12) Patent Application Publication
(10) Pub. No.: US 2025/0345299 A1
(43) Pub. Date: Nov. 13, 2025

(54) COMBINATION AND COMPOSITION COMPRISING NICOTINIC ACID OR DERIVATIVE THEREOF AND FATTY ACID OR DERIVATIVE THEREOF

(71) Applicant: T OMEGA AS, Tananger (NO)

(72) Inventor: Rolf Kristian BERGE, Bønes (NO)

(73) Assignee: T OMEGA AS, Tananger (NO)

(21) Appl. No.: 18/864,812

(22) PCT Filed: Apr. 17, 2023
(86) PCT No.: PCT/NO2023/050084

§ 371 Date: Nov. 11, 2024

(30) Foreign Application Priority Data
May 10, 2022 (NO) 20220533

(51) Int. Cl. A61K 31/19; A61K 31/706

(52) U.S. Cl. CPC A61K 31/19; A61K 31/706

(57) ABSTRACT

The invention relates to combination or composition comprising: (i) nicotinic acid or a derivative thereof, and (ii) a fatty acid or a derivative thereof selected from tetradecylthioacetic acid 5 (TTA) or a derivative thereof. The invention further relates to a pharmaceutical or nutritional combination or composition for use in a method of preventing or treating mitochondrial diseases, inflammatory diseases, metabolic disorders, neurodegenerative diseases and/or aging of an animal, and/or a method of increasing brain metabolism of an animal, wherein said pharmaceutical or nutritional 10 combination or composition is administered to said animal, and wherein said pharmaceutical or nutritional combination or composition comprises: (i) nicotinic acid or a derivative thereof, and (ii) a fatty acid or a derivative thereof selected from tetradecylthioacetic acid (TTA) or a derivative thereof.

### FIELD OF THE INVENTION

The present invention is directed to a combination and composition comprising nicotinic acid or a derivative thereof, and a fatty acid or a derivative thereof. More specifically, the present invention relates to such a combination and composition as well as uses thereof for, inter alia, improving mitochondrial function and/or reducing inflammation and/or reducing metabolic disorders in the body of an animal, and preventing or treating mitochondria-related diseases, inflammatory diseases and/or metabolic disorders, and preventing or treating neurogenerative diseases, neurodegeneration or aging and/or strengthening an energy flux between liver and brain of an animal, including humans.

### BACKGROUND OF THE INVENTION

Fatty acids are vital for all living organisms. They are integrated constituents of all types of cell membranes and are involved in many ways in the generation and storage of energy, in modifying membrane structure and function and in regulating cellular signaling pathways.

Disorders of lipid metabolism are intimately connected to many common, lifestyle-related diseases or so called metabolic syndrome, see FIG. 1. The manifestations of many of these disorders can be influenced by the diet.

Mitochondrial diseases are a group of metabolic disorders the symptoms of which may vary, depending on how many mitochondria are defective and where they are in the body. Sometimes only one organ, tissue, or cell type is affected. But often the problem affects many of them. Muscle and nerve cells have especially high energy needs, so muscular and neurological problems are common. The diseases range from mild to severe. Some types can be fatal.

Other metabolic disorders include obesity, type 2 diabetes, pre-diabetes, and metabolic syndrome. The global health crisis of such disorders is well established. The prevalence of each one of them is reaching pandemic proportions world-wide and their prevalence is expected to continue to rise in the next decades, thereby further exacerbating the current world wide health crisis surrounding these diseases.

The beneficial effects of marine oils are well established although the health effects are variable dependent on the source of oils, the metabolic disorders to be addressed and which population to be targeted (age, gender, lifestyle, etc.). Omega-3 fatty acids and other dietary compounds have been found to affect the hepatic mitochondrial function in some animals, and the use thereof may increase the mitochondrial fatty acid oxidation, a process for burning long-chain fatty acids. Investigations of the effects of dietary lipids and other dietary compounds have further demonstrated their effects on insulin and glucose homeostasis, plasma lipids, fat depots and anti-oxidant and anti-inflammatory activities, including reduced plaque formation in mice aorta and improved health status in inflammatory intestines. Studies have further shown that omega-3 fatty acids and other dietary compounds in combinations have positive effects on lipid metabolism and indicate that they potentiate the lipid lowering effects of marine oils (fish, krill) and improve mitochondrial function.

Although a wide range of compounds and compositions are known to be useful in improving the function of different organs of the body, and in preventing or treating several of these disorders and diseases, there is still a need for new and improved compounds and compositions that can be used in preventing, reducing, alleviating or reversing mitochondrial dysfunction and/or improving mitochondrial function in the body of animals, including humans. There is also still a need for new and improved compounds and compositions that can be used in preventing, reducing, alleviating or reversing inflammation in the body of animals, including humans. Further, there is also a need for new and improved compounds and compositions that can be used in preventing, reducing, alleviating or reversing metabolic disorders of animals, including humans. Finally, there is a need for new and improved compounds that can be used in preventing, reducing, alleviating or reversing the reduced energy gap during neurodegeneration and aging in animals, including humans, and strengthening the energy flux between the liver and brain, e.g. ketones from the liver and substances that can penetrate the blood-brain barrier.

### SUMMARY OF THE INVENTION

It is an object of the invention to provide a combination, composition and compounds that are effective in preventing, reducing, alleviating or reversing mitochondrial dysfunction and/or improving mitochondrial function in the body of an animal.

It is another object of the invention to provide a combination, composition and compounds that are effective in preventing, reducing, alleviating or reversing inflammation in the body of an animal.

It is yet another object of the invention to provide a combination, composition and compounds that are effective in preventing, reducing, alleviating or reversing metabolic disorders of an animal.

It is yet another object of the invention to provide a combination, composition and compounds that can be used in preventing, reducing, alleviating or reversing the reduced energy gap during neurodegeneration and aging in animals, including humans, and strengthening the energy flux between the liver and brain, e.g. ketones from the liver and substances that can penetrate the blood-brain barrier.

It is yet another object of the invention to provide a pharmaceutical or nutritional combination, composition and compounds that can be used in preventing or treating mitochondrial diseases, inflammatory diseases, metabolic disorders, neurodegenerative diseases and/or aging in an animal.

The present invention provides combinations, compositions and compounds that can be used to prevent or treat serious conditions and/or diseases of an animal, such as mitochondrial diseases, inflammatory diseases and/or metabolic disorders. Examples of such mitochondrial diseases include diseases that are caused by or associated with mitochondrial dysfunction in one or more organs of the body of an animal, i.e., mitochondrial diseases, or mitochondria-related diseases, such as for example neurodegenerative diseases (NDs), e.g. Parkinson's disease (PD), Huntington's disease (HD), dementia, Alzheimer's disease (AD), and amyotrophic lateral sclerosis (ALS); cancer, diabetes, including type 1 diabetes, type 2 diabetes (T2D), gestational diabetes and maternally inherited diabetes and deafness (MIDD), kidney diseases, lung diseases and cardiovascular diseases, including atherosclerosis. Examples of inflammatory diseases that can be prevented or treated according to the present invention include chronic inflammatory diseases, also referred to as autoimmune diseases. Examples of metabolic disorders that can be prevented or treated according to the present invention include metabolic syndrome (MS), obesity, e.g. abdominal obesity, type 2 diabetes (T2D), and pre-diabetes.

According to the invention, the combinations, compositions and compounds of the invention provide improved effects, usually synergistic effects, i.e. the effects of the combinations, compositions and compounds are greater than, or provide benefits that are different than, the effects of each of the individual compounds of the combinations and compositions when administered alone.

Hereby the present invention makes it possible to achieve prevention, inhibition, reduction, treatment, relief and/or reversal of organ dysfunction, organ lesions, (chronic) inflammations, conditions and/or diseases, milder symptoms of conditions and/or diseases, and improvement in health, overall well-being and/or increased life quality.

Accordingly, in one aspect, the present invention relates to a combination or composition comprising:
(i) nicotinic acid or a derivative thereof, and
(ii) a fatty acid or a derivative thereof.

In yet another aspect, the present invention relates to a pharmaceutical or nutritional combination or composition for use in a method of preventing or treating mitochondrial diseases, inflammatory diseases, metabolic disorders, neurodegenerative diseases and/or aging of an animal, wherein the pharmaceutical or nutritional combination or composition is administered to the animal, and wherein the pharmaceutical or nutritional combination or composition comprises:
(i) nicotinic acid or a derivative thereof, and
(ii) a fatty acid or a derivative thereof.

In yet another aspect, the present invention relates to a method of preparing a composition comprising mixing:
(i) nicotinic acid or a derivative thereof, and
(ii) a fatty acid or a derivative thereof.

In yet another aspect, the present invention relates to a method of preventing or treating a mitochondrial disease, inflammatory disease, metabolic disorder, neurodegenerative disease and/or aging, which comprises administering to a subject in need thereof a combination or composition comprising:
(i) nicotinic acid or a derivative thereof, and
(ii) a fatty acid or a derivative thereof.

In yet another aspect, the present invention relates to a method of preventing or treating a reduced energy gap during or caused by neurodegeneration or aging, strengthening an energy flux between liver and brain and/or increasing metabolism of a brain, the which comprises administering to a subject in need thereof a combination or composition comprising:
(i) nicotinic acid or a derivative thereof, and
(ii) a fatty acid or a derivative thereof.

In other aspects, the present invention relates to a combination, composition, pharmaceutical combination, pharmaceutical composition, nutritional combination and nutritional composition, as defined herein, as well as methods and uses, as defined herein, comprising:
(i) nicotinic acid or a derivative thereof, and
(ii) a fatty acid or a derivative thereof selected from tetradecylthioacetic acid (TTA) or a derivative thereof.

These and other objects and aspects of the invention will be described in further detail hereinafter.

### BRIEF DESCRIPTION OF THE DRAWINGS

FIG. 1 is an illustration of the cardiometabolic syndrome and life-style related diseases.

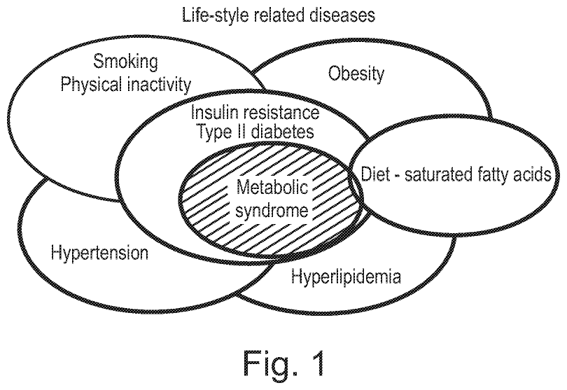

FIG. 2 is an illustration of the choline oxidation pathway taking place in mitochondria.

FIG. 3 is an illustration of the tryptophan-kynurenine and tryptophan-nicotinamide pathways.

FIG. 4 is an illustration of the tryptophan-nicotinamide pathway.

FIG. 5 is an illustration of oleic acid metabolism in brain cells of rats when using compounds of the invention compared to when using the individual compounds alone.

### DETAILED DESCRIPTION OF THE INVENTION

The present invention generally relates to a combination or composition comprising:
(i) nicotinic acid or a derivative thereof, and
(ii) a fatty acid or a derivative thereof.

According to a preferred embodiment of the invention, the fatty acid or a derivative thereof is tetradecylthioacetic acid (TTA) or a derivative thereof, preferably tetradecylthioacetic acid (TTA).

Further, the present invention generally relates to a pharmaceutical or nutritional combination or composition for use in a method of preventing or treating mitochondrial diseases, inflammatory diseases, metabolic disorders, neurodegenerative diseases and/or aging of an animal, and/or for use in a method of increasing the metabolism of the brain of an animal, wherein said pharmaceutical or nutritional combination or composition is administered to said 35 animal, and wherein the pharmaceutical or nutritional combination or composition comprises:
(i) nicotinic acid or a derivative thereof, and
(ii) a fatty acid or a derivative thereof.

In a preferred embodiment of the invention, the fatty acid or a derivative thereof is tetradecylthioacetic acid (TTA) or a derivative thereof, preferably tetradecylthioacetic acid (TTA).

The term “compound” and “compounds”, as used herein, refers to a chemical compound and two or more chemical compounds, respectively. The term “compounds of the invention”, as used herein, refers to the nicotinic acid or a derivative thereof and the fatty acid or a derivative thereof, which are included or comprised in the combinations and compositions of the invention. The term “combination”, as used herein, refers to two or more chemical compounds or agents, which may be separate, e.g. used separately, or together, e.g. in the form of a composition. The term “composition”, as used herein, refers to a mixture of two or more chemical compounds or agents. The term “agent”, as used herein, refers a chemical compound or a mixture of two or more chemical compounds. The term “combinations of the invention”, as used herein, is meant to include the combination of the invention, the pharmaceutical combination of the invention, and the nutritional combination of the invention, unless otherwise stated. The term “compositions of the invention”, as used herein, is meant to include the composition of the invention, the pharmaceutical composition of the invention, and the nutritional composition of the invention, unless otherwise stated. The term “combinations and compositions of the invention”, as used herein, is meant to include both the combinations of the invention and the compositions of the invention, unless otherwise stated.

The term “pharmaceutical composition”, as used herein, refers to a composition comprising one or more compounds or agents that are pharmaceutically active for animals or humans, and/or a composition comprising an active pharmaceutical ingredient (API) that produces one or more intended effects of the body of an animal or human.

The term “nutritional composition”, as used herein, refers to a composition comprising any ingestible material, including but not restricted to nutritional supplements, functional foods, herbal supplements etc. for animal or human consumption. The term also includes food products for human consumption and animal fodder, wherein the composition of the present invention is an additive, and not the main ingredient. This especially concerns animal fodder, where any fodder can be supplemented with the nutritional composition of the invention, to attain the biological effects thereof.

The terms “prevent”, “preventing” and “prevention”, as used herein, include precluding, reducing the risk of developing and delaying the onset of a medical condition or one or more symptoms or complications associated with the condition. The terms “treat”, “treating” and “treatment”, as used herein, include alleviating, ameliorating, inhibiting the progress of, reversing or abrogating a medical condition or one or more symptoms or complications associated with the condition, and alleviating, ameliorating or eradicating one or more causes of the condition. Reference to “treatment” of a medical condition includes prevention of the condition. The term “medical condition” (or “condition” for brevity), as used herein, includes one or more diseases and disorders. The terms “disease” and “disorder” are used interchangeably herein.

Examples of suitable nicotinic acid and derivatives thereof according to the invention include nicotinic acid (also known as niacin, and vitamin B3), amides and esters thereof, including nicotinamide (NAM) (also referred to as niacinamide) and derivatives thereof, including nicotinic riboside compounds, including nicotinamide riboside (NR) and nicotinamide mononucleotide (NMN), including derivatives of both the oxidized form and the reduced form of NR (NRH), nicotinic acid riboside (NAR), including derivatives of both the oxidized form and the reduced form of NAR, (NARH) and derivatives thereof, nicotinamide adenine dinucleotide (NAD) and derivatives thereof, including both the oxidized form and the reduced form of NAD, including NAD+ and NADH, nicotinates, including methyl nicotinate (nicotinic acid methyl ester), niceritrol, 2-aminopyridine-3-carboxylic acid, 5-aminopyridine-3-carboxylic acid, methyl 2-fluouo-6-(pyrrolidon-1-yl) nicotinate, 2-chloro-6-methylnicotinic acid, 5-Hydroxy-2-(2-methyl-3-trifluoromethylanilino) nicotinic acid, and combinations thereof. Examples of preferred nicotinic acid and derivatives thereof according to the invention include nicotinic acid, nicotinamide (NAM), nicotinamide riboside (NR), nicotinamide mononucleotide (NMN), nicotinic acid riboside (NAR), nicotinates, and combinations thereof, e.g. nicotinamide riboside (NR), nicotinic acid riboside (NAR), and combinations thereof.

The fatty acid or derivative thereof according to the invention may be represented by
a) the general formula R″—COO—(CH₂)₂ₙ₊₁—X—R′, wherein X is a sulphur atom, a selenium atom, an oxygen atom, a CH₂ group, a SO group or a SO₂ group; n is an integer of 0 to 11; and R′ is a linear or branched alkyl group, saturated or unsaturated, optionally substituted, wherein the main chain of said R′ contains from 13 to 23 carbon atoms and optionally one or more heterogroups selected from the group consisting of an oxygen atom, a sulphur atom, a selenium atom, an oxygen atom, a CH₂ group, a SO group and a SO₂ group; and R″ is a hydrogen atom or an alkyl group containing from 1 to 4 carbon atoms;
   and/or
b) the general formula (I),

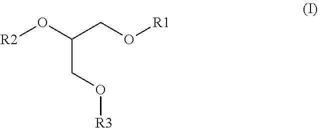

wherein R1, R2, and R3 represent
i) a hydrogen atom; or
ii) a group having the formula CO—R in which R is a linear or branched alkyl group, saturated or unsaturated, optionally substituted, and the main chain of said R contains from 1 to 25 carbon atoms; or
iii) a group having the formula CO—(CH₂)₂ₙ₊₁—X—R′, wherein X is a sulphur atom, a selenium atom, an oxygen atom, a CH₂ group, a SO group or a SO₂ group; n is an integer of 0 to 11; and R′ is a linear or branched alkyl group, saturated or unsaturated, optionally substituted, wherein the main chain of said R′ contains from 13 to 23 carbon atoms and optionally one or more heterogroups selected from the group consisting of an oxygen atom, a sulphur atom, a selenium atom, an oxygen atom, a CH₂ group, a SO group and a SO₂ group;
iv) an entity selected from the group consisting of PO₃CH₂CHNH₃COOH (serine), iv) PO₃CH₂CH₂NH₃ (ethanolamine), PO₃CH₂CH₂N(CH₃)₃ (choline), PO₃CH₂CHOHCH₂OH (glycerol) and PO₃(CHOH)₆ (inositol);
wherein R1, R2, and R3 are selected independently from i), ii), iii), or iv), but at least one of R1, R2, or R3 is defined by iii); and/or
c) the general formula (II),

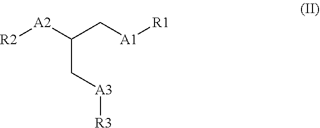

wherein A1, A2 and A3 are selected independently and represent an oxygen atom, a sulphur atom or an N—R4 group in which R4 is a hydrogen atom or a linear or branched alkyl group, saturated or unsaturated, optionally substituted, containing from 1 to 5 carbon atoms;
wherein R1, R2, and R3 represent
i) a hydrogen atom or a linear or branched alkyl group, saturated or unsaturated, optionally substituted, containing from 1 to 23 carbon atoms; or
ii) a group having the formula CO—R in which R is a linear or branched alkyl group, saturated or unsaturated, optionally substituted, and the main chain of said R contains from 1 to 25 carbon atoms; or
iii) a group having the formula CO—(CH₂)₂ₙ₊₁—X—R′, wherein X is a sulphur atom, a selenium atom, an oxygen atom, a CH₂ group, a SO group or a SO₂ group; n is an integer of 0 to 11; and R′ is a linear or branched alkyl group, saturated or unsaturated, optionally substituted, wherein the main chain of said R′ contains from 13 to 23 carbon atoms and optionally one or more heterogroups selected from the group consisting of an oxygen atom, a sulphur atom, a selenium atom, an oxygen atom, a CH₂ group, a SO group and a SO₂ group;
iv) an entity selected from the group consisting of —PO₃CH₂CHNH₃COOH (serine), PO₃CH₂CH₂NH₃ (ethanolamine), PO₃CH₂CH₂N(CH₃)₃ (choline), PO₃CH₂CHOHCH₂OH (glycerol) and PO₃(CHOH) 6 (inositol);
wherein R1, R2, and R3 are selected independently from i), ii), iii), or iv), but at least one of R1, R2, or R3 is defined by iii); and/or a salt, prodrug or complex of the compounds according to a) to c).

Examples of suitable fatty acids and derivatives thereof according to the invention include those in which:
at least one of said R1, R2 and R3 is an alkyl group;
at least one of said R1, R2 and R3 is an alkene group;
at least one of said R1, R2 and R3 is an alkyne group;
said X is a sulphur atom or a selenium atom, preferably a sulphur atom;
said n is 0 or 1;
at least one of said R1, R2 and R3 is tetradecylthioacetic acid;
at least one of said R1, R2 and R3 is tetradecylselenoacetic acid; and/or the fatty acid or derivative thereof has a chain, or main chain, which is saturated, unsaturated, or polyunsaturated, and which contains from 13 to 23 carbon atoms and optionally one or more heterogroups selected from the group consisting of an oxygen atom, a sulphur atom, a selenium atom, an oxygen atom, an SO group and an SO₂ group.

Examples of preferred fatty acids and derivatives thereof according to the invention include those in which at least one of said R1, R2 and R3 is tetradecylthioacetic acid or a tetradecylthioacetic acid group.

Examples of suitable fatty acids and derivatives thereof include non β-oxidizable fatty acids, tetradecylthioacetic acid (TTA), tetradecylselenoacetic acid, TTA containing one or more carbon-carbon triple bonds, e.g. 2-(tridec-12-yn-1-ulthio) acid, 3-thia-15-heptadecyne, as well as esters thereof, including mono-, di- and tri-esters thereof, mono-, di, and tri-acylglycerides thereof, amides thereof, including mono-, di- and tri-amides thereof, peptides thereof; compounds comprising a phospholipid, including phospholipids selected from the group consisting of phosphatidyl serine, phosphatidyl choline, phosphatidyl ethanolamine, phosphatidyl inositol, phosphatidyl glycerol, diphosphatidyl glycerol; the phosphatidylcholine (PC) derivative 1,2-ditetradecylthioacetoyl-sn-glycero-3-phosphocholine, the phosphatidylethanolamine (PE) derivative 1,2-ditetradecylthioacetoyl-sn-glycero-3-phosphoethanolamine; mono-, di, and tri-acylglycerides of fatty acids; polyunsaturated fatty acids in the form of a mono-, di- or triglyceride, ester, e.g. ethyl ester, free fatty acid or salt thereof; omega-3, omega-6 and/or omega-9 polyunsaturated fatty acids in the form of a mono-, di- or triglyceride, ester, e.g. ethyl ester, free fatty acid or salt thereof; eicosapentaenoic acid (EPA) and docosahexaenoic acid (DHA) in the form of a mono-, di- or triglyceride, ester, e.g. ethyl ester, free fatty acid or salt thereof, and combinations thereof.

Examples of preferred fatty acids and derivatives thereof include non β-oxidizable fatty acids and derivatives thereof, tetradecylthioacetic acid (TTA) and derivatives thereof, including their derivatives as defined above; mono-, di, and tri-acylglycerides of fatty acids, including the fatty acids as defined above; polyunsaturated fatty acids and derivatives thereof, including their derivatives as defined above, as well as EPA and DHA and their derivatives as defined above, e.g. tetradecylthioacetic acid (TTA) and derivatives thereof, and combinations thereof.

During β-oxidation, a fatty acid is enzymatically oxidized cleaved between carbons 2 and 3 (when counting from the carboxylic end of the fatty acid), resulting in the removal of the two carbon atoms on either side of the oxidation site as acetic acid. This step is then repeated on the now two carbons shorter fatty acid, and repeated again until the fatty acid is fully oxidized. β-oxidation is the usual way in which the majority of fatty acids are catabolized in vivo. The β-oxidation blocking by some of the compounds of the invention is achieved by the insertion of a non-oxidizable group in the X position in the above formulae, in which X is defined as S, O, SO, SO₂, CH₂ or Se. Fatty acids containing S, O, SO, SO₂, CH₂ or Se in the X position in the above formulae are referred to as non β-oxidizable fatty acids.

The term “mitochondrial diseases”, as used herein, also referred to as “mitochondria-related diseases”, refers to disorders caused by dysfunctional mitochondria, or mitochondrial dysfunction, and may occur when the mitochondria of the cell fail to produce enough energy for cell or organ function. A mitochondrial disease can be due to, e.g., a congenital genetic deficiency or an acquired deficiency. A mitochondrial disease can be caused by, e.g., oxidative damage during aging, elevated intracellular calcium level, exposure of affected cells to nitric oxide, ischemia, hypoxia, microtubule-associated deficit in axonal transport of mitochondria, or expression of mitochondrial uncoupling proteins. Congenital mitochondrial diseases result from hereditary mutations, deletions or other defects in mitochondrial DNA, in nuclear genes regulating mitochondrial DNA integrity, or in nuclear genes encoding proteins. Acquired mitochondrial defects can be caused by, e.g., damage to mitochondrial DNA due to oxidative processes or aging, mitochondrial dysfunction, inhibition of respiratory chain complexes, mitochondrial respiration defects and deficiencies, oxygen deficiency, impaired nuclear-mitochondrial interactions, and expression of mitochondrial uncoupling proteins in response to, e.g., lipids, oxidative damage or inflammation.

Mitochondrial dysfunction plays an important role in several neurological disorders. The pathogenesis and clinical manifestations arise from the fundamental role of bioenergetics in cell biology. Eventually, cells will die if depleted of ATP. Mitochondrial injury may lead to the release of pro-apoptotic factors, e.g. cytochrome c. Many of the pathways involving mitochondrial dysfunction in one mitochondrial disease are also prevalent in the pathogenesis of other mitochondrial diseases.

Examples of diseases that can be prevented or treated according to the present invention include diseases that are caused by or associated with mitochondrial dysfunction in one or more organs of the body of an animal, i.e., mitochondrial diseases, or mitochondria-related diseases, such as for example neurodegenerative diseases (NDs), e.g. Parkinson's disease (PD), Parkinson's disease dementia (PDD), Huntington's disease (HD), dementia, Alzheimer's disease (AD), and amyotrophic lateral sclerosis (ALS); cancer, diabetes, including type 1 diabetes, type 2 diabetes (T2D), gestational diabetes and maternally inherited diabetes and deafness (MIDD), kidney diseases, lung diseases and cardiovascular diseases, including atherosclerosis. Further examples of diseases that can be prevented or treated according to the present invention include inflammatory diseases, including chronic inflammatory diseases, also referred to as autoimmune diseases, and metabolic disorders.

Cancer is a disease caused by an uncontrolled division of abnormal cells in one or more parts of the body of an animal. The abnormal (cancer) cells have a different metabolism than normal cells, which allows them to survive longer and grow faster. Resistance to apoptosis is a key feature of cancer cells, and given the importance of mitochondria in apoptosis, the relationship between cancer development and mitochondrial dysfunction has been manifested on several occasions and in many ways. Accordingly, cancer is regarded as mitochondria-related disease.

Mitochondrial dysfunction can lead also to pancreatic islet β-cell dysfunction, e.g. impaired insulin disfunction and β-cell failure, as well as insulin resistance in various parts of a body. Therefore, mitochondrial dysfunction may cause diabetes, including type 1 diabetes, type 2 diabetes, gestational diabetes and maternally inherited diabetes and deafness (MIDD), also referred to as mitochondrial diabetes, or adversely affect the condition of a diabetic subject.

The kidney is a mitochondria-rich organ in the body. Abnormalities and dysfunction of renal mitochondria may affect several cellular pathways, which may lead to increased oxidative stress, apoptosis, microvascular loss and fibrosis, all of which can impair kidney function.

Mitochondrial dysfunction is a feature of early and persistent lung disease. In particular, mitochondria are central to the pathological processes and clinical phenotypes associated with a wide range of lung diseases. An abnormal mitochondrial function plays an important role in many lung diseases, e.g. asthma, cystic fibrosis, chronic obstructive pulmonary disease (COPC), lung cancer, pneumonia, tuberculosis, pulmonary arterial hypertension, and idiopathic pulmonary fibrosis (IPF).

Mitochondrial dysfunction is also involved in the pathogenesis of several cardiovascular diseases, including myocardial infarction, cardiomyopathies of various etiologies, arrhythmias, atherosclerosis, and hypertension. Since the heart is highly dependent on mitochondrial oxidative energy, defects in mitochondrial structure and function can be found in association with different cardiac diseases. As used herein, the term “cardiovascular diseases” (CVDs) refer disorders of the heart and blood vessels. Further examples of cardiovascular diseases include coronary heart disease, cerebrovascular disease, peripheral arterial disease, rheumatic heart disease, deep vein thrombosis and pulmonary embolism, also referred to as venous thromboembolism, and arteriosclerosis, which is a corona artery disease (CAD). Arteriosclerosis is characterized by inflammation, macrophage invasion, foam cell formation, intima thickening, accretion of cholesterol and formation of atherosclerotic plaque, which over time can become calcified. Atherosclerosis is invariably associated with inflammation and starts with destruction of the endothelium at the luminal side of the tunica intima. The onset of atherosclerosis is invariably in the large arteries such as for example, the aorta and coronary arteries. In more advanced stages there may be plaque rupture leading to sudden vascular occlusion, myocardial infarction and cerebrovascular accident (infarction of the brain).

The term “inflammatory diseases”, as used herein, refers to disorders caused by agents, such as bacteria and viruses, where binding of specific antibodies to the agents causes cascade reactions and gives rise to inflammatory reactions. When the agents are eliminated, the inflammation is down-regulated and the tissue will reverse to normal. Inflammatory diseases also comprise chronic inflammatory diseases which refer to conditions where the inflammation remains even in the absence of such agents. “Chronic inflammatory diseases” are inflammatory diseases caused by immune-reactions towards a subject's own proteins, i.e., an autoimmune reaction, and the conditions are also referred to as “autoimmune diseases”. The term “chronic” means that the inflammatory disease persists or lasts for an extended period of time, e.g. for years or throughout life.

Examples of autoimmune diseases that can be prevented or treated according to the invention include acquired hemophilia, acute motor axonal neuropathy, Addison's disease, adult-onset Still's disease, alopecia areata, ankylosing spondylitis, anti-glomerular basement membrane nephritis, anti-neutrophil cytoplasmic antibody-associated vasculitis, anti-N-methyl-D-aspartate receptor encephalitis, anti-sperm antibodies, antiphospholipid syndrome, anti-synthetase syndrome, aplastic anemia, autoimmune angioedema, autoimmune encephalitis, autoimmune enteropathy, autoimmune gastritis, autoimmune hemophilia, autoimmune hepatitis, autoimmune inner ear disease, autoimmune lymphoproliferative syndrome, autoimmune neutropenia, autoimmune oophoritis, autoimmune orchitis, autoimmune pancreatitis, autoimmune polyendocrine syndrome type 1, autoimmune polyendocrine syndrome type II, autoimmune polyendocrine syndrome type 3, autoimmune progesterone dermatitis, autoimmune retinopathy, autoimmune thrombocytopenia purpura, autoimmune thyroiditis, autoimmune urticaria, autoimmune uveitis, balo concentric sclerosis, Behçet's disease, Bickerstaff's encephalitis, brachial neuropathy (Parsonage-Turner syndrome), Bullous pemphigoid, celiac disease, chronic fatigue syndrome, chronic inflammatory demyelinating polyneuropathy, Churg-Strauss syndrome, cicatricial pemphigoid, Cogan syndrome, cold agglutinin disease, complex regional pain syndrome, CREST syndrome, Crohn's disease, cryptogenic organizing pneumonia, cutaneous lupus erythematosus, dermatitis herpetiformis, dermatomyositis, diabetes mellitus type 1, diffuse interstitial keratitis, discoid lupus erythematosus, endometriosis, enthesitis-related arthritis, eosinophilic esophagitis, eosinophilic fasciitis, epidermolysis bullosa acquisita, episcleritis, encephalopathy associated with autoimmune thyroid disease, erythema nodosum, essential mixed cryoglobulinemia, Evans syndrome, Felty syndrome, fibromyalgia, gestational pemphigoid, giant cell arteritis, goodpasture syndrome, Graves' disease, Graves ophthalmopathy, Guillain-Barre syndrome, Hashimoto's Encephalopathy, Hashimoto Thyroiditis, hidradenitis suppurativa, idiopathic dilated cardiomyopathy, idiopathic pulmonary fibrosis, IgA nephropathy, IgG4-related systemic disease, inclusion body myositis, inflammatory bowel diseases (IBD), intermediate uveitis, interstitial cystitis, juvenile arthritis, Kawasaki's disease, Lambert-Eaton myasthenic syndrome, leukocytoclastic vasculitis, lichen planus, lichen sclerosus, ligneous conjunctivitis, linear IgA disease, lupus nephritis, lupus vasculitis, Lyme disease (chronic), Menière's disease, microscopic colitis, microscopic polyangiitis, mixed connective tissue disease, Mooren's ulcer morphea, Mucha-Habermann disease, multiple sclerosis, myasthenia gravis, myelin oligodendrocyte glycoprotein disease (MOG), myocarditis, myositis, narcolepsy with cataplexy, neuromyelitis optica, neuromyotonia opsoclonus myoclonus syndrome, optic neuritis, Ord's thyroiditis, palindromic rheumatism, paraneoplastic cerebellar degeneration, Parry Romberg syndrome, Parsonage-Turner syndrome, pediatric autoimmune neuropsychiatric disorder associated with streptococcus, pemphigus vulgaris, pernicious anemia, pityriasis lichenoides et varioliformis acuta, POEMS syndrome, polyarteritis nodosa, polymyalgia rheumatica, polymyositis postmyocardial infarction syndrome, postpericardiotomy syndrome, primary biliary cirrhosis, primary sclerosing cholangitis, psoriasis, including guttate, inverse, erythrodermic and pustular psoriasis, psoriatic arthritis (PsA), pure red cell aplasia, purpura rheumatica, pyoderma gangrenosum, Raynaud's phenomenon, reactive arthritis, relapsing polychondritis, restless leg syndrome, retinocochleocerebral vasculopathy, retroperitoneal fibrosis, rheumatic chorea, rheumatic fever, rheumatoid arthritis (RA), rheumatoid vasculitis, sarcoidosis, Schnitzler syndrome, scleritis, scleroderma, Sjogren's syndrome, stiff person syndrome, subacute bacterial endocarditis, Susac's syndrome, Sydenham chorea, sympathetic ophthalmia, systemic lupus erythematosus (SLE), systemic scleroderma, Takayasu arteritis, Tolosa-Hunt syndrome, transverse myelitis, ulcerative colitis (UC), undifferentiated connective tissue disease, urticaria, urticarial vasculitis, vasculitis, vitiligo, warm autoimmune hemolytic anemia, autoimmune cholestatic liver diseases such as primary biliary cholangitis (PBC) and primary sclerosing cholangitis (PSC), and combinations thereof, including multiple autoimmune syndrome, i.e. the combination of at least three autoimmune diseases (MAS).

The term “metabolic disorders”, as used herein, refers to disorders associated with aberrant whole-body glucose, lipid and/or protein metabolism of an animal and pathological consequences arising therefrom, including metabolic syndrome (MS), type 2 diabetes, obesity, and pre-diabetes, which are also associated with the risk of developing cardiovascular disease.

Examples of metabolic disorders that can be prevented or treated according to the invention include metabolic syndrome, obesity, e.g. abdominal obesity, and prediabetes. Key elements of the metabolic disorders include impaired fasting glucose or impaired glucose tolerance, increased waist circumference, elevated fasting plasma glucose, increased fasting plasma triglycerides, decreased fasting high density lipoprotein level, increased blood pressure, insulin resistance, hyperinsulinemia, cardiovascular disease (or components thereof such as arteriosclerosis, coronary artery disease, peripheral vascular disease, or cerebrovascular disease), congestive heart failure, elevated plasma norepinephrine, elevated cardiovascular-related inflammatory factors, elevated plasma factors potentiating vascular endothelial dysfunction, hyperlipoproteinemia, arteriosclerosis or atherosclerosis, hyperthermia, hyperphagia, hyperglycemia, hyperlipidemia, and hypertension or high blood pressure, increased plasma postprandial triglyceride or free fatty acid levels, increased cellular oxidative stress or plasma indicators thereof, increased circulating hypercoagulative state, hepatic steatosis, renal disease including renal failure and renal insufficiency, liver diseases, including fatty liver diseases, metabolic dysfunction associated fatty liver disease (MAFLD), also known as non-alcoholic fatty liver disease (NAFLD), including non-alcoholic fatty liver (NAFL) and non-alcoholic steatohepatitis (NASH).

Examples of preferred metabolic disorders that can be prevented or treated according to the invention include obesity, obesity-related complications, hypertension, cardiovascular disease, nephropathy, elevated plasma glucose concentrations, type 2 diabetes (T2D), type 1 diabetes, hyperglycemia, insulin resistance, hyperthermia and fatty liver diseases.

The prevention and/or treatment according to the invention is suitably carried out for a period of time during which the condition and/or disease, or the symptoms, manifestations and complications thereof, persist. As the symptoms, manifestations and complications may persist for weeks, months, or years, the period of time for the prevention, treatment and/or administration may be at least 1 week, or at least 2 weeks, or at least 4 weeks, or at least 3 months, or at least 12 months, or at least 18 months, or at least 36 months, or for the rest of the life of the subject.

The combinations, compositions and compounds of the invention may comprise one or more additional pharmaceutically acceptable components, including one or more pharmaceutically acceptable inactive components, e.g. carriers, excipients and diluents, and/or one or more pharmaceutically acceptable active components.

The term “pharmaceutically acceptable”, as used herein, refers to a substance (e.g., an inactive or active component, ingredient or excipient) that is suitable for use in contact with the tissues and organs of a subject without excessive irritation, allergic response, immunogenicity and toxicity, is commensurate with a reasonable benefit/risk ratio, and is effective for its intended use. A “pharmaceutically acceptable” excipient or carrier of a pharmaceutical composition is also compatible with the other ingredients of the composition.

Examples of suitable pharmaceutically acceptable inactive components include inert diluents such as carbonates, e.g. calcium carbonate and sodium carbonate; buffers, e.g. phosphate such as calcium phosphate and sodium phosphate, citrates, succinates, acetic acid, and other organic acids and their salts; antioxidants such as ascorbic acid; hydrophilic polymers, e.g. polyvinylpyrrolidone and polyethylene glycols; granulating and disintegrating agents, e.g. corn starch or alginic acid; binding agents, e.g. starch, gelatine or acacia; lubricating agents, e.g. magnesium stearate, stearic acid and talc; solvents, e.g. organic solvents, water and aqueous electrolyte solutions; preservatives; chelating agents, e.g. EDTA; effervescing agents; natural or artificial sweeteners, e.g. glucose, mannose, dextrins, mannitol and sorbitol; carbohydrates, e.g. monosaccharides, disaccharides and other cellulose derivatives; flavouring agents; colouring agents; taste masking agents; acidulants; lactose, surfactants and emulsifiers, e.g. polysorbates, poloxamers, and polyethylene glycols (PEG); thickening agents; suspending, dispersing and wetting agents; oils; waxes; fibers; fats; proteins; carbohydrates; minerals; agents for the improvement of taste, texture, colour, smell, stability and/or storage life; and the like; and combinations thereof.

Examples of suitable pharmaceutically acceptable active components include vitamins, e.g. vitamin A, vitamin C, vitamin D, including vitamin D₁, vitamin D₂ (ergocalciferol, or calciferol), vitamin D₃ (cholecalciferol) and vitamin D analogues, e.g. alfa-calcidol (or 1-hydroxy-cholecalciferol), dihydrotachysterol (DHT) and calcitriol (also known as 1,25-dihydroxy-cholecalciferol); vitamin E and vitamin K; antibiotics; anticoagulants, e.g. acetylsalicylic acid and COX-2 inhibitors; oils; fermented soy protein materials; peptides; carnitine; astraxanthin; and the like; and combinations thereof.

The combinations, compositions and compounds of the invention may be administered to the animal, or subject in need thereof, by various routes, e.g. one or more of the nasal, ophthalmic, oral, enteral, intragastric, sublingual, buccal, rectal, topical, transdermal, inhalational, pulmonary, parenteral, intradermal, intra-arterial, intramuscular, intravascular, intravenous, intracardiac, intraperitoneal, intraocular, subcutaneous, intra-articular, intracapsular, subcapsular, intra-orbital, transtracheal, intrasternal, intrathecal, intraspinal and subarachnoid administrations. If administered in combination but separately, either simultaneously or sequentially, the compounds of the invention may be administered by the same or different routes of administration as defined above.

If administered continuously, the compounds of the invention are each typically administered by 1-4 injections per day or by continuous or semi-continuous subcutaneous infusions, for example, using a mini-pump. An intravenous bag solution may also be employed.

For several routes of administration, e.g. for parenteral administration, the compounds of the invention may be prepared by mixing each at the desired degree of purity, in a unit dosage injectable form (solution, suspension, or emulsion), with a pharmaceutically acceptable carrier to provide a composition of the invention.

The composition of the invention may be prepared by contacting the compounds of the invention each uniformly and intimately with one or more liquid carriers or one or more finely divided solid carriers, or both. Preferably, the carrier is a parenteral carrier, more preferably a solution that is isotonic with the blood of the subject recipient. Examples of suitable carriers include aqueous carriers, e.g. water, aqueous solutions, saline, Ringer's solution, dextrose solution, and non-aqueous carriers, e.g. oils, ethyl oleate, and liposomes.

The pharmaceutical and nutritional combination, composition and compounds of the invention may be provided in any form that is suitable for the intended use or administration. The nutritional combination, composition and compounds of the invention are preferably provided for oral administration.

The pharmaceutical combination, composition and compounds of the invention may be provided in the forms of pills, tablets (coated or uncoated), hard or soft capsules, dragees, lozenges, oral solutions, suspensions and dispersions, syrups or sterile preparations, e.g. for parenteral, intradermal, intra-arterial, intramuscular, intravascular, intravenous, intracardiac, intraperitoneal, intraocular, subcutaneous, intra-articular, intracapsular, subcapsular, intra-orbital, transtracheal, intrasternal, intrathecal, intraspinal and subarachnoid administration.

The nutritional combination, composition and compounds of the invention may be provided in the form of food and beverage products as well as pills, tablets (coated or uncoated), hard or soft capsules, dragees, lozenges, oral solutions, suspensions and dispersions, syrups or sterile preparations. Examples of suitable food and beverage products include juice drinks, dairy drinks, powdered drinks, sports drinks, mineral water, soy beverages, hot chocolate, malt drinks, biscuits, bread, crackers, confectioneries, chocolate, chewing-gum, butters, margarines, spreads, yoghurts, breakfast cereals, snack bars, meal replacements, protein powders, desserts, medical nutrition feeds, nutritional supplements and pellets. The nutritional combination, composition and compounds of the invention may be used as an animal feed or part thereof, e.g. a feed for the animals defined above.

The composition and pharmaceutical or nutritional composition of the invention may contain (i) nicotinic acid or a derivative thereof, and (ii) fatty acid, e.g. tetradecylthioacetic acid (TTA), or a derivative thereof, in amounts and proportions that may vary within wide limits. The composition and pharmaceutical or nutritional composition may contain (i) the nicotinic acid or derivative thereof, and (ii) the fatty acid, e.g. tetradecylthioacetic acid (TTA), or derivative thereof in a therapeutically effective amount when intended to be administered in a single administration per day, or half of a therapeutically effective amount when intended to be provided by means of two administrations per day, or 1/x of a therapeutically effective amount when intended to be provided by means of x number of administrations per day. The molar proportion between (i) the nicotinic acid or derivative thereof, and (ii) the fatty acid, e.g. tetradecylthioacetic acid (TTA), or derivative thereof may be from 99:1 to 1:99, usually from 80:20 to 20:80, suitably from 60:40 to 40:60, and preferably round 50:50.

The term “therapeutically effective amount”, as used herein, refers to an amount of an agent or therapeutic agent, or a combination of agents or therapeutic agents, that, when administered to a subject, is sufficient to prevent, reduce the risk of developing, delay the onset of, slow the progression of or cause regression of the medical condition being treated, or to alleviate to some extent the medical condition or one or more symptoms or complications of that condition, at least in some fraction of the subjects taking that agent. The term “therapeutically effective amount” also refers to an amount of an agent or therapeutic agent, or a combination of agents or therapeutic agents, that is sufficient to elicit the biological or medical response of a cell, tissue, organ, system, animal or human which is sought by a researcher, veterinarian, medical doctor or clinician.

The term “therapeutic agent”, as used herein, refers to any biologically, physiologically or pharmacologically active substance that acts locally or systemically in a subject and is administered to a subject for purposes of diagnosis, treatment, mitigation, cure or prevention of a disease or enhancement of a desirable physical or mental development or condition.

The therapeutically effective amount of (i) the nicotinic acid or derivative thereof may correspond to a dosage of from 10 to 2,000 μg/day, or from 50 to 1,500 μg/day, or from 100 to 1,000 μg/day. The therapeutically effective amount of (i) the nicotinic acid or derivative thereof may correspond to a dosage of from 70 to 14,000 μg/week, or from 350 to 10,500 μg/week, or from 700 to 7,000 μg/week.

The therapeutically effective amount of (ii) the fatty acid, e.g. tetradecylthioacetic acid (TTA), or derivative thereof may correspond to a dosage of from 10 to 2,000 μg/day, or from 50 to 1,500 μg/day, or from 100 to 1,000 μg/day. The therapeutically effective amount of (ii) the fatty acid, e.g. tetradecylthioacetic acid (TTA), or derivative thereof may correspond to a dosage of from 70 to 14,000 μg/week, or from 350 to 10,500 μg/week, or from 700 to 7,000 μg/week.

When vitamin D is present in the combinations, compositions and compounds of the invention, the dosage of vitamin D may be from 1 to 500 μg/day, or from 2 to 250 μg/day, or from 5 to 100 μg/day.

The pharmaceutical and nutritional combination, and the method of preventing or treating a mitochondrial disease and/or inflammatory disease according to the invention, may comprise administering a first amount of the nicotinic acid or derivative thereof in combination with a second amount of the fatty acid, e.g. tetradecylthioacetic acid (TTA), or derivative thereof. It should be understood that a “combination” or “in combination”, as used herein, may refer to simultaneous and/or sequential administration. For example, the invention may comprise administering the first amount of nicotinic acid or derivative thereof followed by administering the second amount of fatty acid, e.g. tetradecylthioacetic acid (TTA), or derivative thereof before or during the time that the first amount of nicotinic acid or derivative thereof is (or becomes) active in the body, or vice versa. The method may comprise administering the first amount of nicotinic acid or derivative thereof simultaneously or about simultaneously with the second amount of fatty acid, e.g. tetradecylthioacetic acid (TTA), or derivative thereof.

The first and second amounts may be administered in one or more daily doses. It should be understood that a “dose” refers to the quantity of an agent administered at a particular point in time. The first amount of nicotinic acid or derivative thereof may be administered or delivered in a single daily dose or may be administered or delivered over the course of multiple doses per day. For example, the first amount of nicotinic acid or derivative thereof may be administered in one, two, three, four, five, or more daily doses, wherein each dose contains the same or a different amount of nicotinic acid or derivative thereof with respect to one or more other doses. Similarly, the second amount of fatty acid, e.g. tetradecylthioacetic acid (TTA), or derivative thereof may be administered in one, two, three, four, five, or more daily doses, wherein each dose contains the same or a different amount of fatty acid, e.g. tetradecylthioacetic acid (TTA), or derivative thereof with respect to one or more other doses. Each dose of the nicotinic acid and derivatives thereof may independently be administered or delivered simultaneously with or sequentially with respect to a dose of the fatty acid, e.g. tetradecylthioacetic acid (TTA), or derivative thereof. Further, each dose of the fatty acid, e.g. tetradecylthioacetic acid (TTA), or derivative thereof may be administered simultaneously with or about simultaneously with a dose of the nicotinic acid or derivative thereof.

The amount, or first amount, of the nicotinic acid or derivative thereof may be a therapeutically effective amount, optionally when administered in combination with the fatty acid, e.g. tetradecylthioacetic acid (TTA), or derivative thereof. The therapeutically effective amount of the nicotinic acid or derivative thereof may be an amount thereof that lowers the therapeutically effective amount of the fatty acid, e.g. tetradecylthioacetic acid (TTA), or derivative thereof.

The amount, or second amount, of the fatty acid, e.g. tetradecylthioacetic acid (TTA), or derivative thereof may be a therapeutically effective amount, optionally when administered in combination with the nicotinic acid or derivative thereof. The therapeutically effective amount of the fatty acid, e.g. tetradecylthioacetic acid (TTA), or derivative thereof may be an amount thereof that lowers the therapeutically effective amount of the nicotinic acid or derivative thereof.

The animal according to the invention may be any animal and/or mammal, including fish and shellfish, e.g. salmon, cod, tilapia, clams, oysters, lobster and crabs; amphibians, e.g. frogs; reptiles, e.g. turtles; birds, e.g. chicken such as hens, cockerel and roosters, and turkeys; anatidae, e.g. ducks, geese and swans; felines, e.g. cats; canids, e.g. dogs and wolves; rodents, e.g. mice, rats, hamsters, gerbils and guinea pigs; lagomorphs, e.g. rabbits and hares; camelids, e.g. camels, llamas an alpacas; farm animals, e.g. pigs, sheep, goats, horses, cows, deer and mink; other mammal, e.g. species in zoos and conservation centers not stated above, such as hippopotamus, rhinoceros, etc.; primates, e.g. apes, chimpanzees, monkeys, and humans. Preferably the animal is a mammal, and preferably the mammal is a human. The term “subject” as used herein refers to any animal, including a mammalian subject, e.g. a human subject.

### EXAMPLES

The invention is further illustrated in the following examples which, however, are not intended to limit the same. Parts and % relate to parts by weight and % by weight, respectively, and all suspensions are aqueous, unless otherwise stated.

### Example 1

This example illustrates an animal study using male Wistar rats which were fed NR, NAR, TTA and combinations thereof.

The animal study was conducted according to the Guidelines for the Care and Use of Experimental Animals, and in accordance with the Norwegian legislation and regulations governing experiments using live animals. All efforts were made to optimize the animal environment and minimize suffering. Eight weeks-old old male Wistar rats, Rattus norvegicus, weighing 400-450 g, were purchased from Taconic (Ejby, Denmark). Upon arrival the rats were randomized using Research Randomizer [12], labeled and placed in open cages, two in each cage, where they were allowed to acclimatize to their surroundings for one week. During the acclimatization and experiment period, the rats had unrestricted access to tap water, but restricted access to chow to obtain a constant dose through the experiment.

The rats were kept in a 12 hours light/dark cycle at a constant temperature (22±2° C.) and a relative humidity of 55% (±5%). Upon start of the experiment, the rats were block randomized to their respective interventions. All rats were divided into seven groups. The first group of animals (control group) was fed a high-fat diet (HF) with 25% by weight fat, consisting of 23% by weight lard and 2% by weight soybean oil, referred to as “HF”. The second group was fed a high-fat diet (HF) supplemented with tetradecylthioacetic acid (TTA) (250 mg/day/kg body weight), referred to as “HF+TTA”. The third group was fed a high-fat diet (HF) supplemented with nicotineamide riboside (NR) (100 mg/day/kg body weight), referred to as “HF+NR”. The fourth group was fed a high-fat diet (HF) supplemented with nicotinic acid riboside (NAR) (40 mg/day/kg body weight), referred to as “HF+NAR”. The fifth group was fed a high-fat diet (HF) supplemented with both tetradecylthioacetic acid (TTA) (250 mg/day/kg body weight) and nicotineamide riboside (NR) (100 mg/day/kg body weight), referred to as “HF+TTA+NR”. The sixth group was fed a high-fed diet (HF) supplemented with both tetradecylthioacetic acid (TTA) (250 mg/day/kg body weight) and nicotinic acid riboside (NAR) (40 mg/day/kg body weight), referred to as “HF+TTA+NAR”. The seventh group was fed a low-fed diet, referred to as “LF”.

All animals were weighed daily and feed intake was determined weekly. The animal experiment was run for 12 weeks. At sacrifice, rats were anaesthetized by inhalation of 2-5% isoflurane (Schering-Plough, Kent, UK), the abdomen opened along the midline, and exsanguination was performed using cardiac puncture. EDTA-blood was collected and immediately chilled on ice. The samples were centrifuged and plasma was stored at −80° C. prior to analysis. Different tissues were collected, weighed and were immediately snap-frozen in liquid nitrogen and stored at −80° C. until further analysis.

### Example 2

Analysis of the samples obtained in Example 1 relating to HF, TTA, NR, combinations thereof and LF in terms of compounds relevant to mitochondrial function, immune system, inflammatory reactions, metabolic function and/or cellular processes showed the results set forth in Table 1, where the values are given in μM.

TABLE 1

| | | | | HF + | |
|---|---|---|---|---|---|
| Compound | | HF + | HF + | TTA + | |
| analyzed | HF | TTA | NR | NR | LF |
| Glycine | 259 | 382 | 221 | 385 | 179 |
| 3-Hydroxybutyrate | 160 | 261 | 287 | 314 | 115 |
| Acetoacetate | 49.3 | 91.6 | 72.9 | 123.5 | 39.3 |
| 3-Hydroxyanthranilic | 5.6 | 12.7 | 5.4 | 14.0 | 9.0 |
| acid | | | | | |
| Quinolinic acid | 600 | 2830 | 533 | 3109 | 571 |
| Nicotinic acid | 5.04 | 13.99 | 12.38 | 37.89 | 5.06 |
| Nicotinamide | 2409 | 6340 | 5870 | 10736 | 2634 |
| N1- | 722 | 2910 | 3616 | 4745 | 1204 |
| Methylnicotinamide | | | | | |
| Pyridoxal 5′- | 554.4 | 537.6 | 729.0 | 695.0 | 713.8 |
| phosphate | | | | | |
| Pyridoxal | 363.9 | 513.5 | 369.8 | 598.7 | 586.8 |
| Methylmalonic acid | 0.50 | 1.99 | 3.37 | 6.28 | 0.61 |
| 2-Aminoadipic acid | 1.49 | 1.64 | 1.33 | 1.74 | 1.29 |

### Example 3

Analysis of the samples obtained in Example 1 relating to HF, TTA, NAR, combinations thereof and LF in terms of compounds relevant to mitochondrial function, immune system, metabolic function and/or cellular processes showed the results set forth in Table 2, where the values are given in μM.

TABLE 2

| | | | | HF + | |
|---|---|---|---|---|---|
| Compound | | HF + | HF + | TTA + | |
| Analyzed | HF | TTA | NAR | NAR | LF |
| Serine | 352 | 563 | 325 | 616 | 330 |
| 3-Hydroxyanthranilic acid | 5.6 | 12.7 | 7.8 | 14.2 | 9.0 |
| Nicotinic acid | 5.04 | 13.99 | 11.95 | 34.06 | 5.06 |
| Nicotinamide | 2409 | 6340 | 4308 | 8923 | 2634 |
| N1-Methylnicotinamide | 722 | 2910 | 2414 | 3994 | 1204 |
| Trigonelline | 0.020 | 0.089 | 0.084 | 0.160 | 0.103 |
| Thiamine | 357.1 | 358.1 | 350.0 | 388.7 | 439.9 |
| monophosphate | | | | | |
| Pyridoxal 5′-phosphate | 554.4 | 537.6 | 598.3 | 757.3 | 713.8 |
| Pyridoxal | 363.9 | 513.5 | 384.1 | 667.3 | 586.8 |
| Folic acid | 0.64 | 0.45 | 0.70 | 0.77 | 1.40 |
| Methylmalonic acid | 0.50 | 1.99 | 2.35 | 9.18 | 0.61 |
| 2-Aminoadipic acid | 1.49 | 1.64 | 1.34 | 1.76 | 1.29 |

### Example 4

As is evident from Tables 1 and 2 of Examples 2 and 3, respectively, the amounts of compounds analyzed that are relevant to the mitochondrial, immune system and metabolic functions and/or cellular processes were higher, or significantly higher, in the samples of plasma of the blood of rats which had been fed with a high-fat diet (HF) supplemented with compounds according to the invention (referred to as “HF+TTA+NR” or “HF+TTA+NAR”), compared to the samples of plasma of the blood of rats which had been fed with a high-fat diet (HF) supplemented with TTA (“HF+TTA”), NR (“HF+NR”) or NAR (“TTA+NAR”). In the tests, the combination or composition comprising nicotinic acid or derivative thereof and fatty acid or derivative thereof according to the invention showed an unexpected and synergistic effect or increase of the amount of the compound analyzed.

### Glycine and Serine

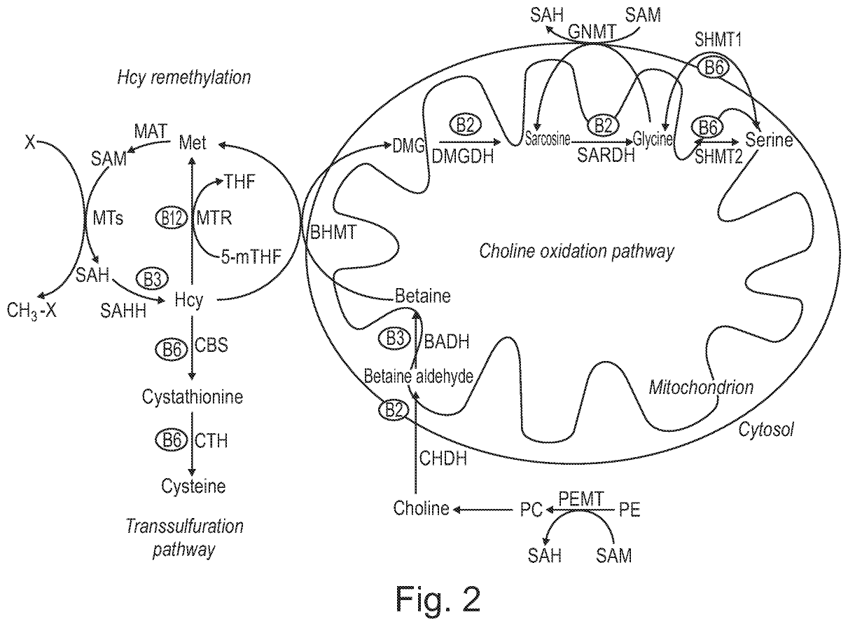

Mitochondrial dysfunction is a key feature in Alzheimer's disease (AD) and it is related to accumulation of amyloid, synaptic failure and neuronal death, processes who correlate with disease progression. Compounds which could stimulate mitochondrial biogenesis and mild mitochondrial uncoupling are promising therapeutic targets in AD. Glycine and serine are metabolites of the choline oxidation pathway that takes place in mitochondria, cf. FIG. 2.

A higher amount of glycine and serine was formed in the mitochondria when feeding rats with a high-fat diet supplemented with both tetradecylthioacetic acid (TTA) and nicotineamide riboside (NR) or nicotinic acid riboside (NAR) according to the invention. The compounds leading to an increased production of glycine and serine can thus be used in preventing, reducing, alleviating or reversing mitochondrial dysfunction, reducing the risk of cognitive dysfunction and development of ischemic stroke. Accordingly, the combination or composition comprising nicotinic acid or derivative thereof and fatty acid or derivative thereof according to the invention can be used in preventing or treating mitochondrial and neurodegenerative diseases.

### 3-Hydroxybutyrate and Acetoacetate

A higher amount of 3-hydroxybutyrate and acetoacetate was formed in the mitochondria when feeding rats with a high-fat diet supplemented with both tetradecylthioacetic acid (TTA) and nicotineamide riboside (NR) according to the invention. 3-Hydroxybutyrate and acetoacetate are ketones and referred to as mitochondrial derived metabolites produced by the mitochondria. Noteworthy, as subjects suffering from Alzheimer's disease (AD) and Parkinson's disease (PD) cannot take up glucose as substrate for ATP formation, they are able to take up ketones and maybe bioactive amino acids to reduce the energy cap created when no glucose can be taken up. The ketones may penetrate the blood-brain barrier and thus represent valuable energy for the brain of subjects suffering from mitochondrial and neurodegenerative diseases such as, for example, Alzheimer's disease (AD). Accordingly, these results show that the combination or composition comprising nicotinic acid or derivative thereof and fatty acid or derivative thereof according to the invention can be used in preventing or treating mitochondrial and neurodegenerative diseases.

### 3-Hydroxyanthranilic Acid and Quinolinic Acid

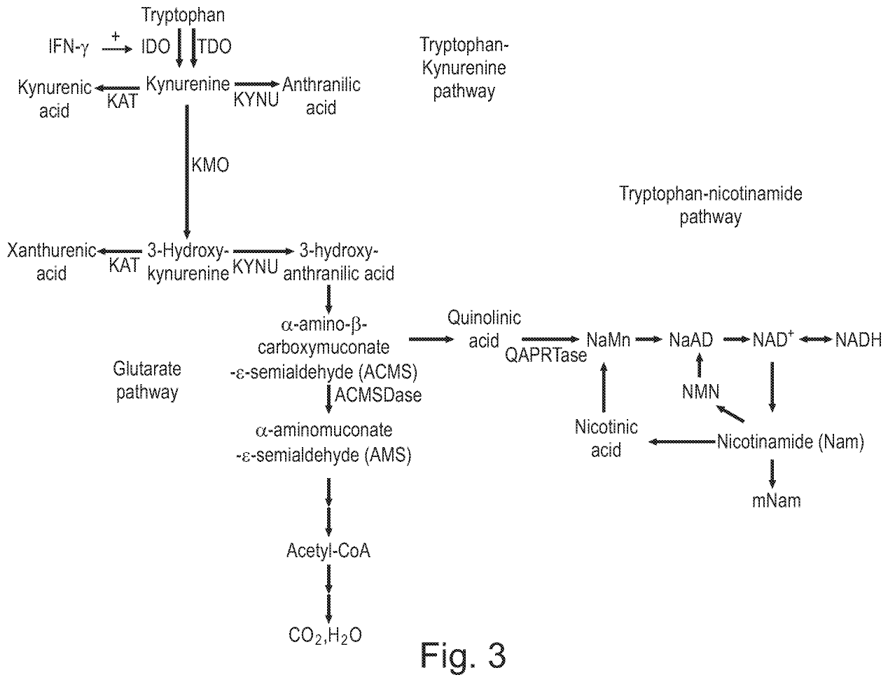

A higher amount of 3-hydroxyanthranilic acid and quinolinic acid was formed when feeding rats with a high-fat diet supplemented with both tetradecylthioacetic acid (TTA) and nicotineamide riboside (NR) according to the invention. 3-Hydroxyanthranilic acid and quinolinic acid are metabolites of the tryptophan-kynurenine pathway, cf. FIG. 3, which is connected to inflammation, the immune system, and neurological conditions. It is the primary route for tryptophan catabolismin the liver and the starting point for the synthesis of nicotinamide adenine dinucleotide (NAD) in animals.

Changes in the plasma level of 3-hydroxyanthranilic acid have been reported to be associated with chronic and acute inflammation and the enzymes responsible for their formation are present in the mitochondria and/or in the cytosol. The results of the tests show that an increased production of 3-hydroxyanthranilic acid obtained by the combination or composition of the invention can be used in the treatment of inflammation and diseases with neurological aspects, especially in cancer patients undergoing chemotherapy. Accordingly, the combination or composition comprising nicotinic acid or derivative thereof and fatty acid or derivative thereof according to the invention can be used in preventing or treating mitochondrial diseases, neurodegenerative diseases, cancer, and inflammatory diseases.

### B3 Vitamers Nicotinic Acid, Nicotinamide, N1-Methylnicotinamide and Trigonelline

B3 vitamers are cofactors for a vast number of enzymatic redox reactions, such as the β-oxidation of fatty acids and substrate oxidation in Krebs cycle, as well as in the synthesis of carnitine. In one-carbon metabolism, vitamin B3 is used as a reducing agent for both MTHFR and MSR, as well as in the conversion of choline to betaine.

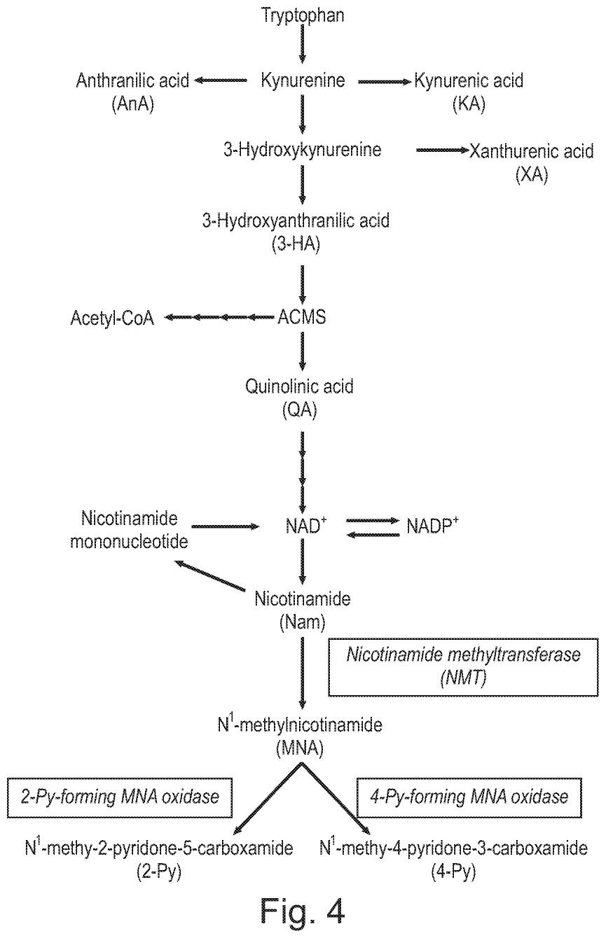

Dysregulation of the tryptophan-nicotinamide pathway has been related to several pathological conditions, and the metabolites in this pathway are known to influence mitochondrial respiration and redox status. B3 vitamers are metabolites of the tryptophan-nicotinamide pathway, cf. FIGS. 3 and 4, and each B3 vitamer is a precursor to NAD+, a substance necessary for cellular energy production. NAD is an essential molecule in all organisms which has two major biological functions. First, it serves as a biochemical catalyst to extract and convert the energy from food sources to enable biological processes. Second, NAD is involved in a multitude of regulatory events that coordinate cellular functions in accordance to the metabolic needs and possibilities. In NAD-dependent regulatory processes, NAD is consumed which necessitates a permanent re-synthesis of the molecule. Over the last years, a number of scientific studies have shown a decline of the NAD concentration associated with aging in general, and with age-associated diseases in particular (including cancer, diabetes and neurodegeneration). Correction of the NAD status can be achieved by nutritional supplementation with intermediates of NAD biosynthesis (NAD supplementation). Restoration or boosting of NAD levels has yielded impressive results in animal models of obesity and diabetes as well as neurodegeneration, which is important in mitochondrial and neurodegenerative diseases such as Alzheimer's disease.

Tables 1 and 2 show that higher amounts of the B3 vitamers nicotinic acid, nicotinamide, N1-methylnicotinamide and trigonelline were formed when feeding rats with a high-fat diet supplemented with tetradecylthioacetic acid (TTA) and either nicotineamide riboside (NR) or nicotinic acid riboside (NAR) according to the invention. The observed higher amounts of the B3 vitamers was accompanied with reduced body weight of the rats, reduced white adipose tissues, plasma lipids and liver lipids, as well as increased amounts of ketones. Accordingly, the increased production of these compounds means increased amounts of intermediates of the NAD biosynthesis (NAD supplementation) and that the combination or composition comprising nicotinic acid or derivative thereof and fatty acid or derivative thereof according to the invention can be used in preventing or treating mitochondrial diseases, neurodegenerative diseases, cancer, diabetes, inflammatory diseases, obesity and metabolic disorders.

### Pyridoxal 5′-phosphate (P5P) and Pyridoxal

Pyridoxal 5′-phosphate and pyridoxal are B6 vitamers, which is a cofactor for approximately 150 reactions that regulate the metabolism of glucose, lipids, amino acids, DNA, and neurotransmitters. Previous reports have showed that vitamin B6 deficiency is associated with both increased oxidative stress and inflammation. Previous reports have also showed an evident inverse association between vitamin B6 levels and diabetes, as well as a clear protective effect of vitamin B6 on diabetic complications, and a protective role of vitamin B6 against diabetes and cancer, e.g. breast cancer of menopausal women. Accordingly, the increased production of these B6 vitamers obtained by the combinations and compositions comprising nicotinic acid or derivative thereof and fatty acid or derivative thereof according to the invention shows that they can be used in preventing or treating mitochondrial diseases, inflammatory diseases, and metabolic disorders.

### 2-Aminoadipic Acid

Previous reports have demonstrated that 2-aminoadipic acid, an intermediate metabolite of lysine metabolism, can modulate insulin secretion and predict type 2 diabetes (T2D). Previous reports have also showed that treatment of diet-induced obesity animals with 2-aminoadipic acid significantly reduced body weight, decreased fat accumulation and lowered fasting glucose. The increased production of 2-aminoadipic acid obtained in the tests show that the combinations or compositions comprising nicotinic acid or derivative thereof and fatty acid or derivative thereof according to the invention can be used in preventing or treating obesity, type 2 diabetes (T2D), mitochondrial, metabolic and endocrine disorders, thus in preventing or treating mitochondrial diseases and metabolic disorders.

### Example 5

Analysis of samples obtained in Example 1 relating to HF, TTA, NR, combinations thereof and LF in terms of parameters and/or substances relevant to mitochondrial function, immune system, inflammatory reactions, metabolic function and/or cellular processes showed the results set forth in Table 3.

TABLE 3

| | | | | HF + | |
|---|---|---|---|---|---|
| Parameter/ | | HF + | HF + | TTA + | |
| substance analyzed | HF | TTA | NR | NR | LF |
| Weights [g] | | | | | |
| Body weight start | 421.30 | 421.50 | 409.60 | 430.10 | 413.80 |
| Body weight end | 581.50 | 543.80 | 555.60 | 541.30 | 569.80 |
| White adipose tissue | 43.07 | 33.54 | 34.52 | 34.03 | 42.82 |
| Brown adipose tissue | 0.316 | 0.400 | 0.308 | 0.427 | 0.320 |
| Plasma lipids [mmol/l] | | | | | |
| Triglycerides | 1.947 | 0.620 | 2.194 | 0.590 | 2.064 |
| Cholesterol | 2.086 | 1.638 | 2.000 | 1.425 | 2.550 |
| High density lipoprotein | 1.100 | 1.150 | 0.938 | 1.000 | 1.775 |
| Low density lipoprotein | 0.500 | 0.388 | 0.450 | 0.300 | 0.400 |
| HDL/LDL ratio | 2.257 | 3.011 | 2.117 | 3.313 | 4.483 |
| Liver lipids [μmol/g] | | | | | |
| Triglycerides | 81.60 | 38.42 | 75.22 | 42.70 | 21.89 |
| Cholesterol | 35.95 | 9.36 | 39.84 | 8.83 | 6.27 |
| Total fatty acids [μg/ml] | | | | | |
| Plasma | 2860 | 1604 | 3049 | 1410 | 3438 |

As is evident from Table 3 of Example 5, the amounts of compounds analyzed that are relevant to the mitochondrial, immune system and metabolic functions and/or cellular processes were better, or significantly better, in samples of rats which had been fed with a high-fat diet (HF) supplemented with compounds according to the invention (referred to as “HF+TTA+NR”), compared to the samples of rats which had been fed with a high-fat diet (HF) supplemented with TTA (“HF+TTA”) or NR (“HF+NR”). In the tests, the combination or composition comprising nicotinic acid or derivative thereof and fatty acid or derivative thereof according to the invention showed an unexpected and synergistic effect in the parameters and/or substances analyzed, including lower body weight at the end of the test (less weight gain) and lower amounts of plasma triglycerides, plasma and liver cholesterol, and total fatty acids in plasma.

### Example 6

This example illustrates the impact of the compounds of the invention (TTA+NR) on brain cell metabolism of rats compared to the individual compounds when used alone. The same type of rats as used in Example 1 were used in this example.

### Cell Culture and Treatments

Brain (SH-SY5Y) cells were maintained in Dulbecco's Modified Eagle's Medium/Nutrient Mixture F-12 Ham (DMEM/F12), supplemented with 10% fetal bovine serum (FBS) heat inactivated, 2 mM L-glutamine, 2 mM L-glutamine, 1% non-essential amino acids, penicillin (50 IU), and streptomycin (50 μg/ml)

For the experiment, brain cells were cultured separately in 96-well CellBIND microplates and maintained at 37° C. in a humidified 5% CO₂ atmosphere. The medium was changed regularly every 2-3 days. When the cells reached 80% confluence, treatment incubation was initiated 24 or 48 hours prior the experiment. Before each experiment, nicotinamide riboside (NR) stock solution was prepared at 100 mM in the cell media. Tetradecylthioacetic acid (TTA) stock solution was prepared at 6 mM in 0.1M NaOH and mix with 2.49 mM bovine serum albumin (BSA), used as a vehicle for TTA. The treatments and final solution concentrations are detailed in Table 4.

TABLE 4

| | Basal conditions | TTA treatments |
|---|---|---|
| Basal | Control (cells media) | 100 μM TTA |
| NR | 0.5 mM NR | 100 μM TTA + 0.5 mM NR |

### Substrate Oxidation Assay

Radioactive substrate oxidation assay was performed to evaluate the impact of TTA and NR on cell metabolism. After the incubation with these compounds and their combination, the media was replaced by [1-¹⁴C] oleic acid (OA) (0.5 μCi/ml, 100 μM) substrates during 4 hours and trapping was performed as previously described Weensaas et al. 2007. [33]. In brief, a 96-well UniFilter® microplate, activated for the capture of CO₂ by addition of 1 M NaOH, was mounted on top of the 96-well plate. After 4 hours, the ¹⁴CO₂ trapped in the filter, and radioactive substrate uptake, was measured by the addition of scintillation fluid (Ultima Gold XR) and counted on a 2450 MicroBeta2 scintillation counter. All results were adjusted for protein content, measured by the Bio-Rad protein assay using a VICTOR™ X4 Multilabel Plate Reader.

### Statistical Analysis

Data are presented as mean±standard error mean (SEM) from at least two-three independent experiments with a minimum of four observations for each condition unless stated otherwise. Statistical analyses were performed using g GraphPad Prism 8.0.1 for Windows. Unpaired t test was used to evaluate differences between control and TTA (*); basal and NR conditions (b); TTA and TTA+NR combinations (#). Differences with p values≤0.05 were considered statistically significant.

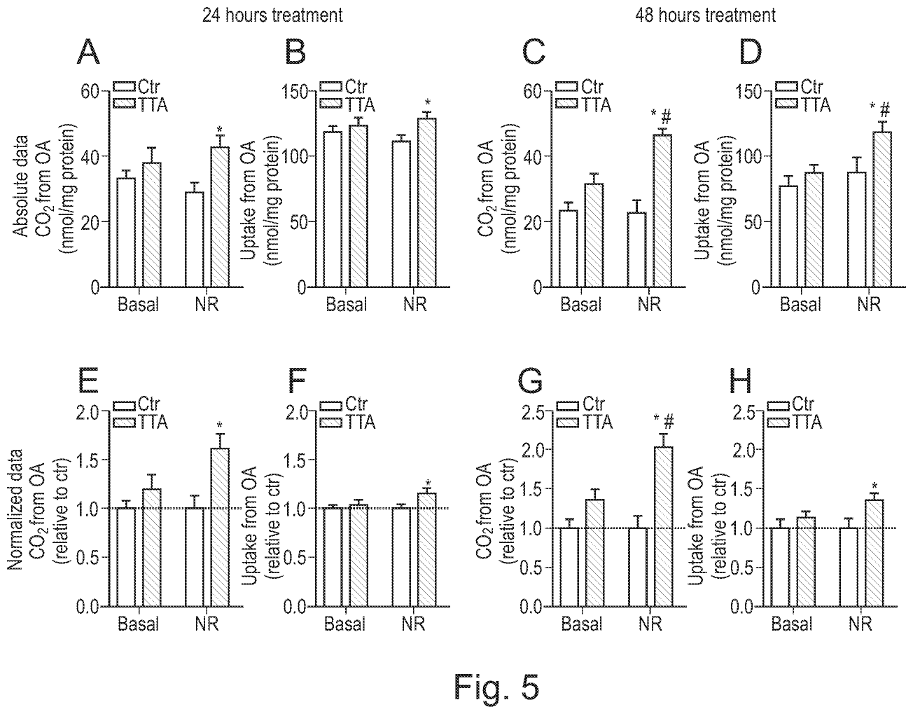

Results are set forth in FIG. 5, which shows oleic acid (OA) metabolism in brain cells (SH-SY5Y). (A-D) Complete OA oxidation (CO₂) and uptake after 24 hours (A-B) and 48 hours (C-D) treatment with 0.5 μM NR and 100 μM TTA alone and in combination (absolute data). (E-H) Effect of TTA treatment in complete OA oxidation (CO₂) and uptake after 24 hours (E-F) and 48 hours (G-H) treatment with 0.5 μM NR and 100 μM TTA alone and in combination (normalized to control (ctr) values). * Statistically significance difference between Ctr and TTA treatments in basal and NR conditions (* p≤0.05 unpaired t test). #Statistically significance difference between TTA (basal) and TTA+NR treatments (#p≤0.05 unpaired t test).

### CONCLUSIONS

As illustrated in this example, the compounds of the invention (TTA+NR) had a significant impact on the metabolism in brain cells of rats compared to when using the individual compounds TTA and NR alone. When used in combination, the compounds of the invention resulted in increased metabolism of oleic acid in brain cells and increased the CO₂ production. Hereby the combination or composition comprising nicotinic acid or derivative thereof and fatty acid or derivative thereof according to the invention can be used in preventing or treating mitochondrial diseases adversely affecting the brain such as, for example, neurodegenerative diseases, e.g. Alzheimer's disease (AD) and Parkinson's disease (PD), and in preventing or treating neurodegeneration or aging of an animal, e.g. by reduced the energy gap caused by neurodegeneration or aging, and/or increasing metabolism of the brain of an animal, e.g. strengthening the energy flux between the liver and brain.

1. A combination or composition comprising:
(i) nicotinic acid or a derivatives thereof, and
(ii) a fatty acid or a derivative thereof selected from tetradecylthioacetic acid (TTA) or a derivative thereof.

2. A pharmaceutical or nutritional combination or composition for use in a method of preventing or treating mitochondrial diseases, inflammatory diseases, metabolic disorders, neurodegenerative diseases and/or aging of an animal, and/or a method of increasing brain metabolism of an animal, wherein said pharmaceutical or nutritional combination or composition is administered to said animal, and wherein said pharmaceutical or nutritional combination or composition comprises:
(i) nicotinic acid or a derivative thereof, and
(ii) a fatty acid or a derivative thereof selected from tetradecylthioacetic acid (TTA) or a derivative thereof.

3. The pharmaceutical or nutritional combination or composition for use in a method according to claim 2, wherein said mitochondrial diseases are selected from the group consisting of neurodegenerative diseases (NDs), Parkinson's disease (PD), Huntington's disease (HD), dementia, Alzheimer's disease (AD), amyotrophic lateral sclerosis (ALS), cancer, type 1 diabetes, type 2 diabetes, gestational diabetes, maternally inherited diabetes and deafness (MIDD), kidney diseases, lung diseases and cardiovascular diseases, atherosclerosis, and combinations thereof.

4. The pharmaceutical or nutritional combination or composition for use in a method according to claim 2, wherein said inflammatory diseases are selected from the group consisting of chronic inflammatory diseases, autoimmune diseases, and combinations thereof.

5. The pharmaceutical or nutritional combination or composition for use in a method according to claim 2, wherein said metabolic disorders are selected from the group consisting of metabolic syndrome, obesity, abdominal obesity, prediabetes, and combinations thereof, preferably said metabolic disorders include disorders selected from the group consisting of impaired fasting glucose, impaired glucose tolerance, increased waist circumference, increased fasting plasma glucose, increased fasting plasma triglycerides, decreased fasting high density lipoprotein (HDL) level, increased blood pressure, insulin resistance, hyperinsulinemia, cardiovascular disease, arteriosclerosis or atherosclerosis, coronary artery disease, peripheral vascular disease, cerebrovascular disease, congestive heart failure, elevated plasma norepinephrine, elevated cardiovascular-related inflammatory factors, elevated plasma factors potentiating vascular endothelial dysfunction, hyperlipoproteinemia, hyperphagia, hyperglycemia, hyperlipidemia, hypertension or high blood pressure, increased plasma postprandial triglyceride or free fatty acid levels, increased cellular oxidative stress or plasma indicators thereof, increased circulating hypercoagulative state, hepatic steatosis, renal disease, renal failure, renal insufficiency, and combinations thereof.

6. The pharmaceutical or nutritional combination for use in a method according to claim 2, wherein said
(i) nicotinic acid or a derivative thereof, and
(ii) a fatty acid or a derivative thereof selected from tetradecylthioacetic acid (TTA) or a derivative thereof,
are administered separately, either simultaneously or sequentially.

7. The pharmaceutical or nutritional combination for use in a method according to claim 2, wherein said animal is selected from the group consisting of fish and shellfish, including salmon, cod, tilapia, clams, oysters, lobster and crabs; amphibians, including frogs; reptiles, including turtles; birds, including chicken, hens, cockerel, roosters and turkeys; anatidae, including ducks, geese and swans; felines, including cats; canids, including dogs and wolves; rodents, including mice, rats, hamsters, gerbils and guinea pigs; lagomorphs, including rabbits and hares; camelids, including camels, llamas and alpacas; farm animals, including pigs, sheep, goats, horses, cows, deer and mink; other mammals, including species in zoos and conservation centers, including hippopotamus, rhinoceros; primates, including apes, chimpanzees, monkeys, and humans, preferably the animal is a mammal, and more preferably the animal or mammal is a human.

8. The combination or composition according to claim 1, or pharmaceutical or nutritional combination or composition for use in a method according to any one of the preceding claims, wherein said (i) nicotinic acid or a derivative thereof is selected from the group consisting of nicotinic acid, nicotinamide (NAM) and derivatives thereof, nicotinic riboside compounds, nicotinamide riboside (NR), nicotinamide mononucleotide (NMN), derivatives of both the oxidized form and the reduced form of NR (NRH), nicotinic acid riboside (NAR), derivatives of both the oxidized form and the reduced form of NAR (NARH), derivatives thereof, NAD+, nicotinates, methyl nicotinate (nicotinic acid methyl ester), niceritrol, 2-aminopyridine-3-carboxylic acid, 5-aminopyridine-3-carboxylic acid, and combinations thereof, preferably said (i) nicotinic acid or a derivative thereof is selected from the group consisting of nicotinic acid, nicotinamide (NAM), nicotinamide riboside (NR), nicotinamide mononucleotide (NMN), nicotinic acid riboside (NAR), nicotinates, and combinations thereof, more preferably said (i) nicotinic acid or a derivative thereof selected from the group consisting of nicotinamide riboside (NR), nicotinic acid riboside (NAR), and combinations thereof.

9. The combination or composition according to claim 1, wherein said (ii) tetradecylthioacetic acid (TTA) or a derivative thereof is represented by
a) the general formula R″—COO—(CH₂)₂ₙ₊₁—X—R′, wherein X is a sulphur atom, a selenium atom, an oxygen atom, a CH₂ group, a SO group or a SO₂ group; n is an integer of 0 to 11; and R′ is a linear or branched alkyl group, saturated or unsaturated, optionally substituted, wherein the main chain of said R′ contains from 13 to 23 carbon atoms and optionally one or more heterogroups selected from the group consisting of an oxygen atom, a sulphur atom, a selenium atom, an oxygen atom, a CH₂ group, a SO group and a SO₂ group; and R″ is a hydrogen atom or an alkyl group containing from 1 to 4 carbon atoms; and/or
b) the general formula (I), 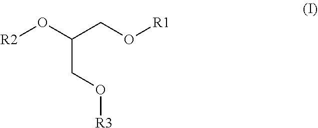
wherein R1, R2, and R3 represent
i) a hydrogen atom; or
ii) a group having the formula CO—R in which R is a linear or branched alkyl group, saturated or unsaturated, optionally substituted, and the main chain of said R contains from 1 to 25 carbon atoms; or
iii) a group having the formula CO—(CH₂)₂ₙ₊₁—X—R′, wherein X is a sulphur atom, a selenium atom, an oxygen atom, a CH₂ group, a SO group or a SO₂ group; n is an integer of 0 to 11; and R′ is a linear or branched alkyl group, saturated or unsaturated, optionally substituted, wherein the main chain of said R′ contains from 13 to 23 carbon atoms and optionally one or more heterogroups selected from the group consisting of an oxygen atom, a sulphur atom, a selenium atom, an oxygen atom, a CH₂ group, a SO group and a SO₂ group;
iv) an entity selected from the group consisting of —PO₃CH₂CHNH₃COOH (serine), PO₃CH₂CH₂NH₃ (ethanolamine), PO₃CH₂CH₂N(CH₃)₃ (choline), PO₃CH₂CHOHCH₂OH (glycerol) and PO₃(CHOH)₆ (inositol);
wherein R1, R2, and R3 are selected independently from i), ii), iii), or iv), but at least one of R1, R2, or R3 is defined by iii); and/or
c) the general formula (II), 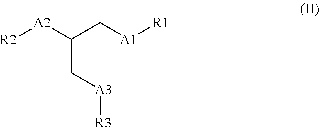
wherein A1, A2 and A3 are selected independently and represent an oxygen atom, a sulphur atom or an N—R4 group in which R4 is a hydrogen atom or a linear or branched alkyl group, saturated or unsaturated, optionally substituted, containing from 1 to 5 carbon atoms;
wherein R1, R2, and R3 represent
i) a hydrogen atom or a linear or branched alkyl group, saturated or unsaturated, optionally substituted, containing from 1 to 23 carbon atoms; or
ii) a group having the formula CO—R in which R is a linear or branched alkyl group, saturated or unsaturated, optionally substituted, and the main chain of said R contains from 1 to 25 carbon atoms; or
iii) a group having the formula CO—(CH₂)₂ₙ₊₁—X—R′, wherein X is a sulphur atom, a selenium atom, an oxygen atom, a CH₂ group, a SO group or a SO₂ group; n is an integer of 0 to 11; and R′ is a linear or branched alkyl group, saturated or unsaturated, optionally substituted, wherein the main chain of said R′ contains from 13 to 23 carbon atoms and optionally one or more heterogroups selected from the group consisting of an oxygen atom, a sulphur atom, a selenium atom, an oxygen atom, a CH₂ group, a SO group and a SO₂ group;
iv) an entity selected from the group consisting of —PO₃CH₂CHNH₃COOH (serine), PO₃CH₂CH₂NH₃ (ethanolamine), PO₃CH₂CH₂N(CH₃)₃ (choline), PO₃CH₂CHOHCH₂OH (glycerol) and PO₃(CHOH)₆ (inositol);
wherein R1, R2, and R3 are selected independently from i), ii), iii), or iv), but at least one of R1, R2, or R3 is defined by iii);
and/or
a salt, prodrug or complex of the compounds according to a) to c).

10. The combination or composition according to claim 9, wherein in said (ii) tetradecylthioacetic acid (TTA) or a derivative thereof, said X is a sulphur atom, and/or said n is 0 or 1, and/or at least one of said R1, R2 and R3 is tetradecylthioacetic acid (TTA) or a tetradecylthioacetic acid group.

11. The combination or composition according to claim 1, wherein said (ii) tetradecylthioacetic acid (TTA) or a derivative thereof is selected from tetradecylthioacetic acid (TTA), TTA containing one or more carbon-carbon triple bonds, including 2-(tridec-12-yn-1-ulthio) acid, 3-thia-15-heptadecyne, as well as mono-, di, and tri-acylglycerides thereof, esters thereof, mono-, di- and tri-esters thereof, amides thereof, mono-, di- and tri-amides thereof, peptides thereof, compounds comprising a phospholipid; the phosphatidylcholine (PC) derivative 1,2-ditetradecylthioacetoyl-sn-glycero-3-phosphocholine, the phosphatidylethanolamine (PE) derivative 1,2-ditetradecylthioacetoyl-sn-glycero-3-phosphoethanolamine; polyunsaturated fatty acids in the form of a di- or triglyceride, esters, salt thereof; omega-3, omega-6 and/or omega-9 polyunsaturated fatty acids in the form of a di- or triglyceride, ester, salt thereof; eicosapentaenoic acid (EPA) and docosahexaenoic acid (DHA) in the form of a di- or triglyceride, or salt thereof; preferably said (ii) tetradecylthioacetic acid (TTA) and derivatives thereof is selected from, mono-, di, and tri-acylglycerides of fatty acids, polyunsaturated fatty acids and derivatives thereof, EPA and DHA and their derivatives, and combinations thereof.

12. The combination or composition according to claim 1, wherein said (ii) tetradecylthioacetic acid (TTA) or a derivative thereof comprise a phospholipid, preferably said phospholipid is selected from the group consisting of phosphatidyl serine, phosphatidyl choline, phosphatidyl ethanolamine, phosphatidyl inositol, phosphatidyl glycerol, and diphosphatidyl glycerol, and mixtures thereof.

13. The combination or composition according to claim 1, wherein it further comprises one or more pharmaceutically acceptable inactive components, preferably the one or more pharmaceutically acceptable inactive components are selected from the group consisting of inert diluents, including carbonates, including calcium carbonate and sodium carbonate; buffers, including phosphate, including calcium phosphate and sodium phosphate, citrates, succinates; organic acids, including acetic acid, and their salts; antioxidants, including ascorbic acid; hydrophilic polymers, including polyvinylpyrrolidone and polyethylene glycols; granulating and disintegrating agents, including corn starch or alginic acid; binding agents, including starch, gelatine and acacia; lubricating agents, including magnesium stearate, stearic acid and talc; solvents, including organic solvents, water and aqueous electrolyte solutions; preservatives; chelating agents, including EDTA; effervescing agents; natural or artificial sweeteners, including glucose, mannose, dextrins, mannitol and sorbitol; carbohydrates, including monosaccharides, disaccharides and cellulose derivatives; flavouring agents; colouring agents; taste masking agents; acidulants; lactose, surfactants and emulsifiers, including polysorbates, poloxamers, and polyethylene glycols (PEG); thickening agents; suspending, dispersing and wetting agents; oils; fibers; fats; proteins; carbohydrates; minerals; agents for the improvement of taste, texture, colour, smell, stability and/or storage life; and combinations thereof.

14. The combination or composition according to claim 1, wherein it further comprises one or more pharmaceutically acceptable active components, preferably the one or more pharmaceutically acceptable active components are selected from the group consisting of vitamins, including vitamin A, vitamin C, vitamin D, including vitamin D₁, vitamin D₂, vitamin D₃ and vitamin D analogues, including alfa-calcidol, dihydrotachysterol and calcitriol; vitamin E, vitamin K; antibiotics; anticoagulants, including acetylsalicylic acid and COX-2 inhibitors; oils; fermented soy protein materials; peptides; carnitine; astraxanthin; and combinations thereof.

15. (canceled)

16. The pharmaceutical or nutritional combination or composition for use in a method according to claim 2, wherein said (ii) tetradecylthioacetic acid (TTA) or a derivative thereof is represented by
a) the general formula R″—COO—(CH₂)₂ₙ₊₁—X—R′, wherein X is a sulphur atom, a selenium atom, an oxygen atom, a CH₂ group, a SO group or a SO₂ group; n is an integer of 0 to 11; and R′ is a linear or branched alkyl group, saturated or unsaturated, optionally substituted, wherein the main chain of said R′ contains from 13 to 23 carbon atoms and optionally one or more heterogroups selected from the group consisting of an oxygen atom, a sulphur atom, a selenium atom, an oxygen atom, a CH₂ group, a SO group and a SO₂ group; and R″ is a hydrogen atom or an alkyl group containing from 1 to 4 carbon atoms; and/or
b) the general formula (I), 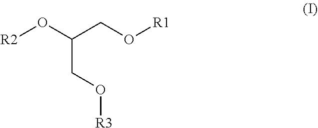
wherein R1, R2, and R3 represent
i) a hydrogen atom; or
ii) a group having the formula CO—R in which R is a linear or branched alkyl group, saturated or unsaturated, optionally substituted, and the main chain of said R contains from 1 to 25 carbon atoms; or
iii) a group having the formula CO—(CH₂)₂ₙ₊₁—X—R′, wherein X is a sulphur atom, a selenium atom, an oxygen atom, a CH2 group, a SO group or a SO2 group; n is an integer of 0 to 11; and R′ is a linear or branched alkyl group, saturated or unsaturated, optionally substituted, wherein the main chain of said R′ contains from 13 to 23 carbon atoms and optionally one or more heterogroups selected from the group consisting of an oxygen atom, a sulphur atom, a selenium atom, an oxygen atom, a CH₂ group, a SO group and a SO₂ group;
iv) an entity selected from the group consisting of —PO₃CH₂CHNH₃COOH (serine), PO₃CH₂CH₂NH₃ (ethanolamine), PO₃CH₂CH₂N(CH₃)₃ (choline), PO₃CH₂CHOHCH₂OH (glycerol) and PO₃(CHOH)₆ (inositol);
wherein R1, R2, and R3 are selected independently from i), ii), iii), or iv), but at least one of R1, R2, or R3 is defined by iii); and/or
c) the general formula (II), 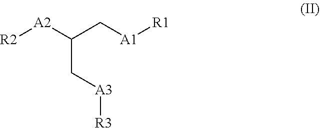
wherein A1, A2 and A3 are selected independently and represent an oxygen atom, a sulphur atom or an N—R4 group in which R4 is a hydrogen atom or a linear or branched alkyl group, saturated or unsaturated, optionally substituted, containing from 1 to 5 carbon atoms;
wherein R1, R2, and R3 represent
i) a hydrogen atom or a linear or branched alkyl group, saturated or unsaturated, optionally substituted, containing from 1 to 23 carbon atoms; or
ii) a group having the formula CO—R in which R is a linear or branched alkyl group, saturated or unsaturated, optionally substituted, and the main chain of said R contains from 1 to 25 carbon atoms; or
iii) a group having the formula CO—(CH₂)₂ₙ₊₁—X—R′, wherein X is a sulphur atom, a selenium atom, an oxygen atom, a CH₂ group, a SO group or a SO₂ group; n is an integer of 0 to 11; and R′ is a linear or branched alkyl group, saturated or unsaturated, optionally substituted, wherein the main chain of said R′ contains from 13 to 23 carbon atoms and optionally one or more heterogroups selected from the group consisting of an oxygen atom, a sulphur atom, a selenium atom, an oxygen atom, a CH₂ group, a SO group and a SO₂ group;
iv) an entity selected from the group consisting of —PO₃CH₂CHNH₃COOH (serine), PO₃CH₂CH₂NH₃ (ethanolamine), PO₃CH₂CH₂N(CH₃)₃ (choline), PO₃CH₂CHOHCH₂OH (glycerol) and PO₃(CHOH)₆ (inositol);
wherein R1, R2, and R3 are selected independently from i), ii), iii), or iv), but at least one of R1, R2, or R3 is defined by iii);
and/or
a salt, prodrug or complex of the compounds according to a) to c).

17. The pharmaceutical or nutritional combination or composition for use in a method according to claim 9, wherein in said (ii) tetradecylthioacetic acid (TTA) or a derivative thereof, said X is a sulphur atom, and/or said n is 0 or 1, and/or at least one of said R1, R2 and R3 is tetradecylthioacetic acid (TTA) or a tetradecylthioacetic acid group.

18. The pharmaceutical or nutritional combination or composition for use in a method according to claim 2, wherein said (ii) tetradecylthioacetic acid (TTA) or a derivative thereof is selected from tetradecylthioacetic acid (TTA), TTA containing one or more carbon-carbon triple bonds, including 2-(tridec-12-yn-1-ulthio) acid, 3-thia-15-heptadecyne, as well as mono-, di, and tri-acylglycerides thereof, esters thereof, mono-, di- and tri-esters thereof, amides thereof, mono-, di- and tri-amides thereof, peptides thereof, compounds comprising a phospholipid; the phosphatidylcholine (PC) derivative 1,2-ditetradecylthioacetoyl-sn-glycero-3-phosphocholine, the phosphatidylethanolamine (PE) derivative 1,2-ditetradecylthioacetoyl-sn-glycero-3-phosphoethanolamine; polyunsaturated fatty acids in the form of a di- or triglyceride, esters, salt thereof; omega-3, omega-6 and/or omega-9 polyunsaturated fatty acids in the form of a di- or triglyceride, ester, salt thereof; eicosapentaenoic acid (EPA) and docosahexaenoic acid (DHA) in the form of a di- or triglyceride, or salt thereof; preferably said (ii) tetradecylthioacetic acid (TTA) and derivatives thereof is selected from, mono-, di, and tri-acylglycerides of fatty acids, polyunsaturated fatty acids and derivatives thereof, EPA and DHA and their derivatives, and combinations thereof.

19. The pharmaceutical or nutritional combination or composition for use according to claim 2, wherein said (ii) tetradecylthioacetic acid (TTA) or a derivative thereof comprise a phospholipid, preferably said phospholipid is selected from the group consisting of phosphatidyl serine, phosphatidyl choline, phosphatidyl ethanolamine, phosphatidyl inositol, phosphatidyl glycerol, and diphosphatidyl glycerol, and mixtures thereof.

20. The pharmaceutical or nutritional combination or composition for use according to claim 2, wherein it further comprises one or more pharmaceutically acceptable inactive components, preferably the one or more pharmaceutically acceptable inactive components are selected from the group consisting of inert diluents, including carbonates, including calcium carbonate and sodium carbonate; buffers, including phosphate, including calcium phosphate and sodium phosphate, citrates, succinates; organic acids, including acetic acid, and their salts; antioxidants, including ascorbic acid; hydrophilic polymers, including polyvinylpyrrolidone and polyethylene glycols; granulating and disintegrating agents, including corn starch or alginic acid; binding agents, including starch, gelatine and acacia; lubricating agents, including magnesium stearate, stearic acid and talc; solvents, including organic solvents, water and aqueous electrolyte solutions; preservatives; chelating agents, including EDTA; effervescing agents; natural or artificial sweeteners, including glucose, mannose, dextrins, mannitol and sorbitol; carbohydrates, including monosaccharides, disaccharides and cellulose derivatives; flavouring agents; colouring agents; taste masking agents; acidulants; lactose, surfactants and emulsifiers, including polysorbates, poloxamers, and polyethylene glycols (PEG); thickening agents; suspending, dispersing and wetting agents; oils; fibers; fats; proteins; carbohydrates; minerals; agents for the improvement of taste, texture, colour, smell, stability and/or storage life; and combinations thereof.

21. The pharmaceutical or nutritional combination or composition for use according to claim 2, wherein it further comprises one or more pharmaceutically acceptable active components, preferably the one or more pharmaceutically acceptable active components are selected from the group consisting of vitamins, including vitamin A, vitamin C, vitamin D, including vitamin D₁, vitamin D₂, vitamin D₃ and vitamin D analogues, including alfa-calcidol, dihydrotachysterol and calcitriol; vitamin E, vitamin K; antibiotics; anticoagulants, including acetylsalicylic acid and COX-2 inhibitors; oils; fermented soy protein materials; peptides; carnitine; astraxanthin; and combinations thereof.
